(19) United States
(12) Plant Patent Application Publication
(10) Pub. No.: US 2014/0013474 P1
(43) Pub. Date: Jan. 9, 2014

(54) Grapevine 'IFG Twelve'

(50) ;

(71) Applicant: David Cain, Bakersfield, CA (US)

(72) Inventor: David Cain, Bakersfield, CA (US)

(73) Assignee: International Fruit Genetics, LLC, Bakersfield, CA (US)

(21) Appl. No.: 13/507,543

(22) Filed: Jul. 9, 2012

(51) Int. Cl. A01H 5/00

(52) U.S. Cl. PLT/206

(57) ABSTRACT

This invention is a new and distinct grapevine variety denominated ‘IFG Twelve’. The new grapevine is characterized by producing very pointed narrow reddish purple grapes having small to medium sized seed traces.

### LATIN NAME OF THE GENUS AND SPECIES CLAIMED

Vitis interspecific hybrid

### VARIETY DENOMINATION

‘IFG Twelve’

### BACKGROUND OF THE INVENTION

The new and distinct grapevine described and claimed herein originated from a hand pollinated cross of the Calinda variety and A2409 an unnamed interspecific very elongate pointed seedless grape selection from the University of Arkansas breeding program hybridized in May 2002. The abortive seed traces were subsequently embryo cultured and the resulting plant was planted in the field in April 2003. The present variety of grapevine was selected as a single plant in July 2004 and was first asexually propagated by hardwood cuttings in December 2004. The resulting propagules were planted during April 2005 near Delano, Kern County, Calif. and were found to reproduce true-to-type through at least two generations of asexual reproduction.

### BRIEF SUMMARY OF THE INVENTION

The new grapevine ‘IFG Twelve’ is characterized by producing extremely narrow elongated seedless fruit with a characteristic pointed tip. The shape, which to the author's knowledge is unique among commercially available seedless table grape varieties, provides consumers with a distinct visual signal to identify the new variety. The new variety is further characterized by producing medium firm reddish black fruits which ripen early in the growing season. Berries are moderately crisp in texture, mild flavored and may have small to medium size rudimentary seed traces. Bunches are long with narrow shoulders and are naturally loose so require no gibberellin for berry thinning. Vines of ‘IFG Twelve’ are moderately productive and may require cane pruning. The fruit ripen early season usually mid to late July in Delano.

‘IFG Twelve’ differs from its maternal parent by producing very elongated narrow pointed reddish black berries having small to medium seed traces as opposed to the red elongated fruits having very large seed traces of the Calinda variety. It differs from its pollen parent by possessing more firm berries with reddish black coloration as opposed to the narrow, elongate pointed black fruits of A2409. The skin of ‘IFG Twelve’ is thinner and the taste is less herbaceous than the A2409.

### BRIEF DESCRIPTION OF THE FIGURE

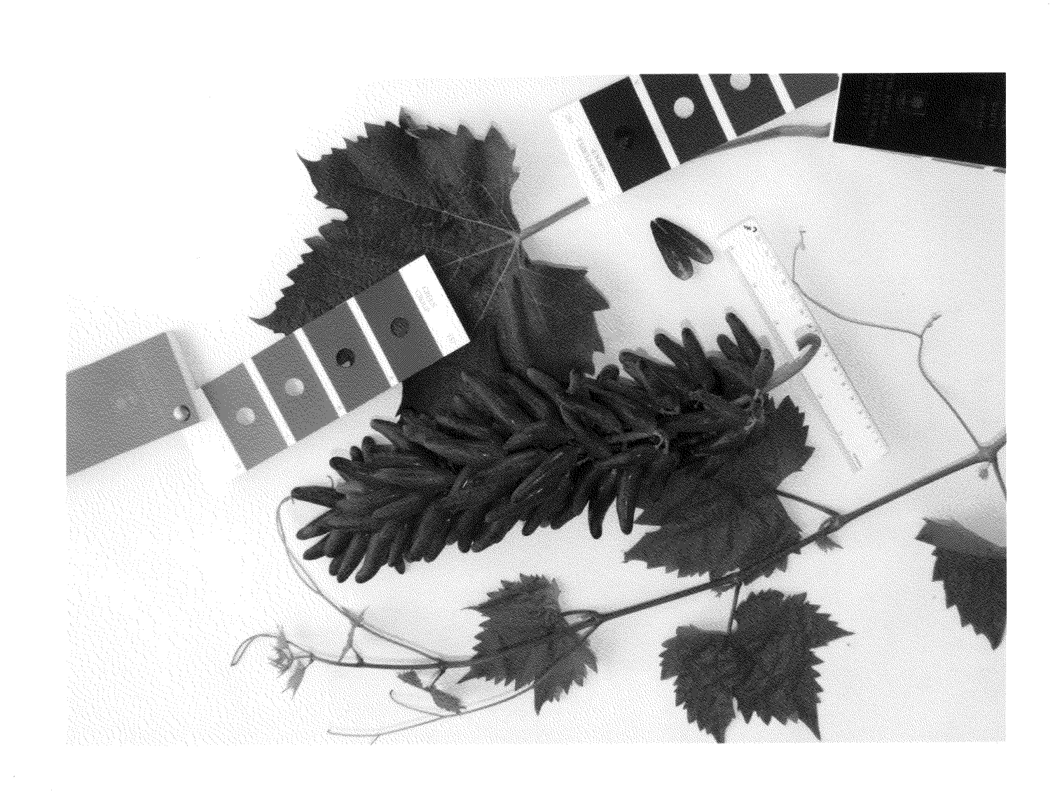

The accompanying photographic illustration in FIG. 1 illustrates in full color ‘IFG Twelve’. The photograph was taken outdoors with indirect lighting. The colors are as nearly true as is reasonably possible in a color representation of this type.

### DETAILED BOTANICAL DESCRIPTION OF THE INVENTION

Throughout this specification, color names beginning with a small letter signify that the name of that color, as used in common speech, is aptly descriptive. Color names beginning with a capital letter designate values based upon R.H.S. Colour Chart, published by The Royal Horticultural Society, London, England.

Throughout this specification subjective description values conform to those set forth by the International Plant Genetic Resources Institute publication ‘Descriptors for Grape’ (vitis spp.) (1983) which was developed in collaboration with the Office International de la Vigne et du Vin (OIV) and the International Union for the Protection of New Varieties of Plants (UPOV).

The descriptive matter which follows pertains to ‘IFG Twelve’ plants grown in the vicinity of Delano, Kern County, Calif. during 2009 and 2010, and is believed to apply to plants of the variety grown under similar conditions of soil and climate elsewhere:
Vine:
   General.—Size — Large Vigor — Vigorous Density of foliage — Dense Productivity — Productive Root stock — Own root Training method — Typically spur pruned leaving 2 bud spurs.
   Trunk.—Trunk diameter of 6-year-old vines at 30 cm above the soil line — 13.1 cm. Shape — Stocky Straps — Very long Surface texture — Shaggy Inner bark color — Grey brown; N199B, Greyed orange; 165A.
Shoots:
   Young shoot.—Form of tip — Wide open Distribution of anthocyanin coloration of tip — Piping (striped) Intensity of anthocyanin coloration of tip — Weak to Medium Density of prostrate hairs of tip — Sparse Density of erect hairs of tip — Absent Color — Yellow- green; N144C, 144B.
   Woody shoot (mature canes).—Shape — Stocky Internode length — Medium; About 10.3 cm Width at node — About 0.8 cm Cross section — Circular Surface — Striate Main color — Greyed — orange; 166A, B, C, 165A, 174A Density of erect hairs of nodes — None or very sparse Density of erect hairs on internodes — None or very sparse Growth of axillary shoots — Strong; Approximately 44.3 cm.
   Flowering shoot.—Vigor during flowering — Strong Attitude during flowering on shoots not tied — Semi-erect Color — dorsal side of internodes — Green with Red stripes Color — ventral side of internodes — Green Color — dorsal side of nodes — Green Color — ventral side of nodes — Green Density of prostrate hairs of nodes — None — Very sparse Density of erect hairs of nodes — None Density of prostrate hairs on internode — None Density of erect hairs on internode — None Anthocyanin coloration of buds — Absent.
   Tendrils.—Distribution on the shoot (at full flowering) — Discontinuous Length of Tendril — Long; About 24.0 cm Thickness — Medium Color — N144A, B, Greyed-orange at tip of new growth; 164A. Form — Bifurcated to trifurcated Number of consecutive tendrils — 2.
Leaves:
   Young leaves.—Color of upper surface of first four distal unfolded leaves — Green with bronze spots Average intensity of anthocyanin coloration of six distal leaves prior to flowering — Absent or very weak Density of prostrate hairs between veins (lower surface) — Absent Density of prostrate hairs on veins (lower surface) — Absent — very sparse Density of erect hairs between veins (lower surface) — Absent Density of erect hairs on veins (lower surface) — Sparse.
   Mature leaves.—Average length — About 16.2 cm Average width — About 14.8 cm Mature leaf size — Medium large Shape of blade — Wedge-shaped Number of lobes — 5 Anthocyanin coloration of main veins on upper side of blade — Very weak Mature leaf profile — V-shaped to undulate Blistering surface of blade upper surface — Very weak Leaf blade tip — In the plane of the leaf Undulation of margin — Slight Thickness — Medium Undulation of blade between main and lateral veins — Slight Shape of teeth — Mixture of both sides straight and both sides convex Length of teeth — Medium Ratio length/width of teeth — Equal Shape of upper lateral sinuses — Open Depth of upper lateral sinuses — Medium General shape petiole sinus — Wide open Shape of base of upper leaf sinuses — U-shaped Tooth at petiole sinus — Present on left side only Density of prostrate hairs between veins on lower surface of blade — Absent Density of erect hairs between veins on lower surface of blade — Absent Density of prostrate hairs on main veins on lower surface of blade — None or very sparse Density of erect hairs on main veins on lower surface of blade — Sparse Density of prostrate hairs on main veins on upper surface of blade — Very sparse — sparse Density of erect hairs on main veins on upper surface of blade — None Autumn coloration of leaves — Greyed purple; n186A, B, 187A, B, Greyed- red 181A, C.
   Upper surface.—Color — Yellow-green to Green; 147A, 137A, B, 146A Anthocyanin coloration of main veins — Absent to very weak Surface appearance — Dull Blistering surface of blade — Absent to Very weak.
   Lower surface.—Color — Yellow green; 147A, 137A, B, 146A. Anthocyanin coloration of main veins (lower surface) — Absent to very weak Glossiness — Weak Surface texture — Smooth Surface appearance — Dull.
   Petiole.—Length — About 11.5 cm Length of petiole compared to middle vein — Slightly shorter Density of prostrate hairs on petiole — None Density of erect hairs on petiole — Sparse.
   Buds.—Bud fruitfulness — Basal: Mostly fruitful Position of first fruitful shoot on previous season cane — 1^{st} to 2^{nd} node Time of bud burst — Medium; Mar. 14, 2011.
Flowers:
   General.—Flower sex — Hermaphrodite Length of first inflorescence — Long; About 29.3 cm long by 6.4 cm wide Position of first flowering and fruiting node — 3^{rd} Number of inflorescence per flowering shoot — 1.1 to 2 Time of bloom — Midseason as compared with similar varieties in the growing area of Delano, Calif. Date of full bloom — May 20, 2011.
Fruit:
   General.—Ripening period — Early to Midseason; Approximately; Jul. 30, 2011 Use — Fresh market Keeping quality — Excellent Resistance to — Insects: Average typical of Vitis vinifera species. Diseases: Average typical of Vitis vinifera species. Refractometer test — solid-sugar: About 20.6 Brix Brix/acid — About 44.8 Titratable acidity — About 0.46 Juice pH — About 4.12.
   Cluster.—Mature cluster length (peduncle excluded) — About 26.1 cm Mature cluster width — About 12.3 cm Mature cluster weight — About 478 g Bunch density — Medium Number of berries — About 204 Form — Cylindrical.
   Peduncle.—Lignification of peduncle — Medium Length of peduncle — Medium. Approximately 4.2 cm.
   Berry.—Uniformity of size — Slightly variable Single berry weight — About 4.4 g natural Shape — Oblong Seeds — Absent Cross section. — Circular Berry dimensions — longitudinal axis: About 40.2 mm. Horizontal axis: About 13.5 mm Berry firmness — Medium Particular flavor — Neutral Bloom (cuticular wax) — Weak Berry separation from pedicel — Difficult Skin color (without bloom) — Greyed-Purple 187A, B.
   Skin.—Thickness — Medium Texture. — Tender Reticulation — Absent Tenacity — Tenacious to flesh.

What is claimed:

1. A new and distinct variety of grapevine as herein illustrated and described.
